(19) United States
(12) Patent Application Publication
(10) Pub. No.: US 2017/0348494 A1
(43) Pub. Date: Dec. 7, 2017

(54) PULMONARY DEVICE, METHOD AND SYSTEM FOR DELIVERING A PHARMACEUTICAL PRODUCT TO AN INDIVIDUAL

(71) Applicants: Nicholas A. Havercroft, Scunthorpe (GB); Donald M. Pell, Saint Petersburg, FL (US); Michael P. Spuza, N. Redington Beach, FL (US); Govindan P. Nair, Saint Petersburg, FL (US)

(72) Inventors: Nicholas A. Havercroft, Scunthorpe (GB); Donald M. Pell, Saint Petersburg, FL (US); Michael P. Spuza, N. Redington Beach, FL (US); Govindan P. Nair, Saint Petersburg, FL (US)

(21) Appl. No.: 15/174,178

(22) Filed: Jun. 6, 2016

(51) Int. Cl. A61M 11/00; A61K 9/08; A24F 47/00; A61K 9/00; A61K 31/465; A61M 15/00

(52) U.S. Cl. CPC A61M 11/001; A61K 31/465; A61M 15/003; A24F 47/002; A61K 9/0078; A61M 15/0021; A61K 9/08; A61M 2205/50; A61M 2205/584; A61M 2205/8206; A61M 2209/10

(57) ABSTRACT

A pulmonary delivery system includes an elongated body having an openable and closeable portion thereof for receiving an insertable and replaceable container such as a cartridge of pure medical product disposed in a saline solution. The medical product may be epinephrine, insulin or nicotine disposed in a saline solution. The system also incorporates a penetrating element that is disposed in the cylinder to pierce the cartridge and release the nicotine solution into the cylinder and into or on a micron mesh grid. A flow of air created by inhaling through a mouthpiece draws air into an individual's lungs from an entrainment port and onto a grid for atomizing a mist of micron droplets through the grid. It is recognized that the air from the entrainment port further dilutes the concentration of the medical product. The grid is connected to a source of electrical power to vibrate the mesh grid.

### FIELD OF THE INVENTION

This invention relates to a pulmonary delivery system and more particularly to a drug delivery system and/or an inhaler for delivering minute amounts of relatively pure but a significant reduced concentrated medical product into an individual's lungs and/or bloodstream.

### BACKGROUND FOR THE INVENTION

Ultrasonic atomizers and nebulizers have been used for many years to atomize relatively small volumes of liquid such as approximately 50 ul to provide a high proportion of lung accessible droplets of less than 10 microns. For example, a U.S. Pat. No. 5,716,002 of Haack et al. issued Feb. 10, 1998 discloses an ultrasonic atomizer which includes an electrically excitable piezoceramic element and a coupling body being operatively connected to the piezoceramic element. The coupling body has a surface coming into contact with a liquid to be atomized and the surface is in the form of a cap-shaped protuberance. The cap-shaped protuberance and the coupling body are formed of a metallic solid material.

A more recent U.S. patent of Ivri et al. U.S. Pat. No. 6,014,970 discloses a method and apparatus for storing chemical compounds in a portable inhaler. As disclosed, there is a cartridge that is coupled to a liquid dispenser and includes a substance in a dry state. A liquid is dispersed from the dispenser into the cartridge so that the substance may be dissolved into the dispersed liquid to form a solution. The solution is then transferred from the cartridge and onto an atomization member which is operated to aerosolize the solution.

Finally, a method for transporting a liquid for atomization and a method and device for atomizing the same are disclosed in a U.S. Pat. No. 9,168,555 of Tsai et al. As disclosed, an ultrasonic nozzle device employing a design of cascaded multiple Fourier horns in resonance produce micrometer-sized monodispersed or narrowly-sized droplets with greatly reduced electrical drive power requirements. The liquid to be atomized is brought externally to or adjacent to the endface of a nozzle tip. The above liquid transport method is equally applicable to the ultrasonic nozzle-array devices that are made up of a plurality of ultrasonic single-nozzle devices configured in parallel. The longitudinal length, transverse width, shape, and area of the nozzle endface of a single-nozzle and nozzle-array devices may be tailored or designed to obtain optimum or large quantities of product droplets to achieve high throughput. By increasing the drive frequency to 8 MHz or higher, sub-micrometer-sized monodispersed or narrowly-sized droplets can be produced using the ultrasonic single-nozzle and nozzle-array devices or any solid endface.

Notwithstanding the above, it is presently believed that there is a need and a potential commercial market for an improved Pulmonary Method and System for Delivering a Pharmaceutical Product in accordance with the present invention.

### SUMMARY OF THE INVENTION

In essence, a first embodiment of the present invention contemplates a pulmonary delivery system for inhaling micron droplets of a liquid medical or pharmaceutical product such as an insulin, epinephrine or nicotine solution into an individual's lungs and into their bloodstream. In a preferred embodiment of the invention, the device or system comprises an atomizer or nebulizer for producing a mass of micron droplets from a vibrating micron mesh grid made from a platinum or palladium plate referred to sometimes herein as “the grid” for producing a mist with particle sizes of five microns or less. In a first form of the invention, the micron mesh grid includes about 4,800 precision holes. The system and device also includes an elongated body which is openable and closeable housing a cylinder for containing a replaceable cartridge.

The system or device also includes an elongated body having an openable and closable portion thereof and/or a cylinder for containing a replaceable cartridge such as a capsule as opposed to a refillable container since refillable containers or cartridges have been strictly avoided in view of the potential risk of spillage and lack of dosage control by a non-professional. A penetrating element is provided for piercing or rupturing the cartridge for delivery of micron droplets to an individual's lungs and into their bloodstream. The device or system also includes a mouthpiece for inhaling micron droplets of a nicotine solution into an individual's lungs, and into their bloodstream and a source of electric energy, and electric circuit for vibrating the micron mesh grid.

A second embodiment of the invention contemplates a portable medical inhaler for limiting the inspiration time of an initial inspiration and subsequently to prevent a second or subsequent inspiration for a preselected time following an initial or previous inspiration. The inhaler includes a tubular body having a cylinder for receiving a cartridge of a limited amount of three and five milliliters of a medical product such as nicotine dissolved in a saline solution. To be more specific, nicotine is addictive and in excess amounts it is a toxic material. It is not generally believed to be a carcinogen. However, the tars and chemicals associated with smoking cigarettes are a carcinogen to a variety of organs and smokers particularly heavy smokers experience withdrawal symptoms within two-to-four hours after a last smoking experience.

However, it has now been recognized that inhaling very small amounts of relatively small particles of a nicotine saline formula can be helpful to avoid withdrawal symptoms. Nevertheless, it is important to prevent the inhaler from delivering an excess amount of nicotine particularly for children and the potential death of pets due to the ingestion of one or more cartridges. Accordingly, an inhaler for emitting an amount of an initial inspiration and to prevent second or subsequent inspirations for a preselected time following an initial or previous inspiration is provided. It is also important to limit the inhaler to use replaceable cartridges as opposed to refillable containers to avoid having relatively untrained individuals working with toxic materials and exposing the nicotine solution to contamination, bacteria, fungus and yeast.

A third embodiment of the invention includes a medical product for reducing and/or eliminating withdrawal symptoms from smoking tobacco products. The product comprises a mixture of between about 97% and about 99 ¾% of a liquid saline solution and from about ¼% and about 3% of a pharmaceutical like grade of nicotine. In addition, the above two compounds can further include an addition of up to about 3% of a flavor. A preferred form of this embodiment includes about 98 ½% saline solution and about 1 ½% of relatively pure nicotine. Like the aforementioned product a preferred form consist essentially of 98 ½% saline solution and about ½%, 1%, 1½% to as much as 3% of nicotine without the tars and chemicals associated with burning or heating tobacco products.

In a fourth embodiment of the invention, a drug delivery system or device includes a microprocessor, a microchip in a circuit for limiting an initial inspiration to two to four seconds of micron droplets and a subsequent or second inspiration prohibited or prevented for a limited period of time such as a period of 15 seconds. This is generally accomplished by timing a discontinuous or prevention of vibration of the micron mesh grid by interrupting a connection between a source of power and the vibrating micron mesh grid. Even though an individual continues to inhale he will receive only air from the entrainment port and/or posterior entrainment port for an inhaled breath in excess of four seconds.

In essence, the present invention contemplates a delivery device, system and method for inhaling micron droplets of a liquid medical product such as a liquid nicotine/saline solution of a reduced concentration into an individual's lungs and into their bloodstream. In its preferred embodiment of the invention the device and/or system comprises an atomizer or nebulizer for producing a mist of micron droplets from a vibrating micron mesh grid with a platinum or palladium surface for producing a mist with particle sizes of two microns or about two microns. In one embodiment of the invention, the micron mesh grid includes 4,800 precision openings in an approximately/inch diameter metal grid. The particle size produced is directly dependent on the size and number of the openings.

The present invention incorporates a platinum or palladium metal grid wherein a converse, inverse or reverse piezoelectric effect i.e. an electric current flowing through the grid causes an atomic realignment of a metal structure to mimic a crystal structure when electricity is passed through the metal grid. Metal has its atomic structure in a random fashion. This is unlike a crystal which has a very strict alignment much like cans lined up in a case with one can on top of another. There are uniform rows and columns of atoms vertically and horizontally. This is why crystals vibrate with a fixed frequency. An example of this is a quartz watch used as a timekeeper. When electric current is applied to a crystal in a watch it vibrates at a constant known frequency. As in the present case of about 128,000 cycles per second the grid vibrates at a frequency of about 128,000 cycles per second.

The device or system in accordance with the present invention includes a tubular body having a cylinder for containing a cartridge containing a nicotine saline solution and wherein the tubular body also includes a penetrating element for piercing or rupturing the cartridge to release a nicotine saline solution on or near the grid made of palladium or platinum metal.

The device or system also includes a mouthpiece for inhaling micron droplets of nicotine solution drawn into an individual's airways and through the alveolar capillary surface of the lungs to enter the individual's bloodstream. In this invention, inhaling a breath of air draws air into the device through the entrainment port much like a venturi system while two 1.5 volt batteries in series use a cellphone type trickle charger to avoid potential problems such as burns, fires and/or explosions by overheated batteries. Such batteries are the sole source of power for vibrating the grid by passing electricity through the grid causing it to vibrate. It is presently believed that the provision of 4,800 holes in the micron mesh grid produces two micron particles.

Further, the device includes a source of electric energy and an electrical circuit powered by two rechargeable nickel cadmium double AA batteries to produce a mist of nicotine solution with particle size of about two micron diameter. The system is recharged by using a cellphone port to plug into a USB connection.

The system also includes a mouthpiece for inhaling micron droplets of atomized nicotine saline solution into the individual's airways and through the alveolar capillary interface into the individual's bloodstream. It is important to maintain a pH between 5.5 to 7 to avoid airway damage and to reduce coughing.

A second embodiment of the invention contemplates a portable medical inhaler or device for limiting the time of an initial inspiration and to prevent a subsequent inhalation for a preselected time following an initial inhalation. The inhaler includes an openable and closeable body housing a cylinder containing a cartridge. To be more specific, nicotine is addictive but is not generally felt to be a carcinogen.

However, it is now known that providing very small amounts of relatively small percentage of a nicotine formulation can be very helpful to stop the craving for a cigarette. Nevertheless, it is important to prevent the inhalation of an excess amount of nicotine particularly where small children may have access to the device. It is also imperative to avoid a possibility that a family pet may eat one or more of these cartridges and become ill.

In a fourth embodiment of the invention, a microchip is included in a circuit that cuts off the flow of electricity to the vibrating micron mesh grid. In this way, the inhalation which is the only source of airflow through the device and into an individual's lungs and beyond and to prevent a second inhalation or puff of the nicotine solution for a period of 15 seconds.

A cartridge contains a limited supply of a liquid pharmaceutical product or modification thereof. In practice, this liquid includes varying concentrations of nicotine ranging from ½% to 3% concentrations of nicotine dissolved in a saline solution. It is also considered that a small amount of flavor of up to about 3% may be included. The inhaler includes a vibratable micron mesh grid that produces an average particle size of two microns. In addition, a source of electric energy, a circuit including a switch for turning off the flow of electricity for energizing the micron mesh grid.

The invention will now be described in connection with the accompanying drawings wherein like numbers are used to indicate like elements.

### DESCRIPTION OF THE DRAWINGS

FIG. 1 is a schematic side view of a drug delivery system in accordance with a first embodiment of the invention;

FIG. 2 is a schematic side view of a forward portion of the drug delivery system illustrated in FIG. 1;

FIG. 3 is a side view of an inner portion of a mouthpiece and drug entrainment port of FIGS. 1 and 2;

FIG. 4 is a schematic side illustration of a portion of a drug delivery system of a further embodiment as shown in FIG. 3; and,

FIG. 5 is a schematic end view of a drug delivery device in accordance with an embodiment of the invention.

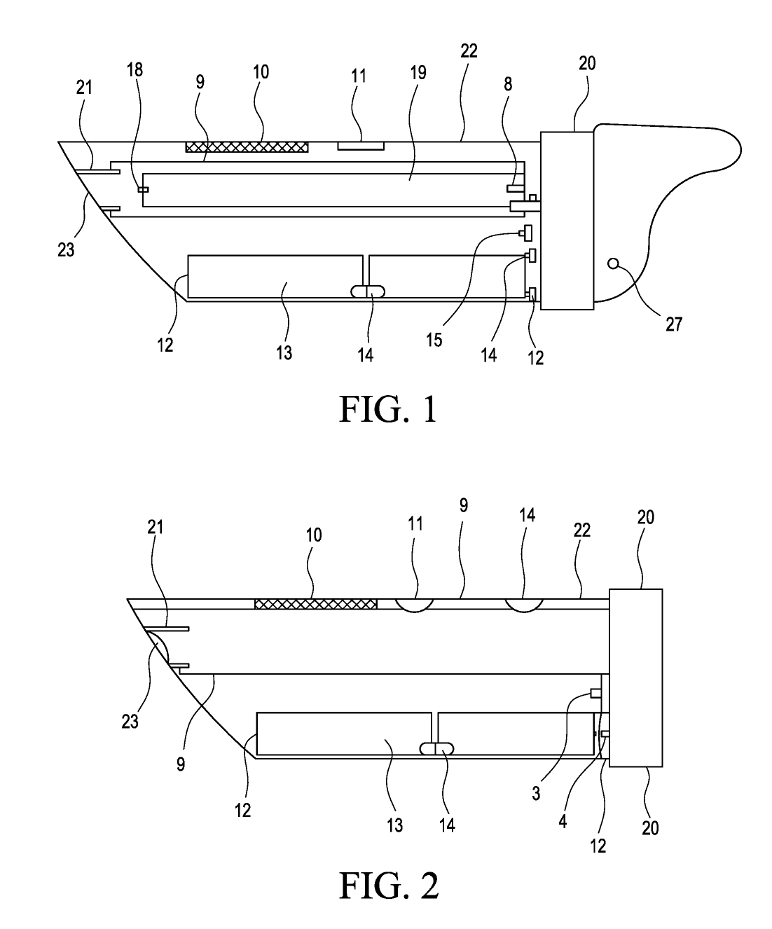

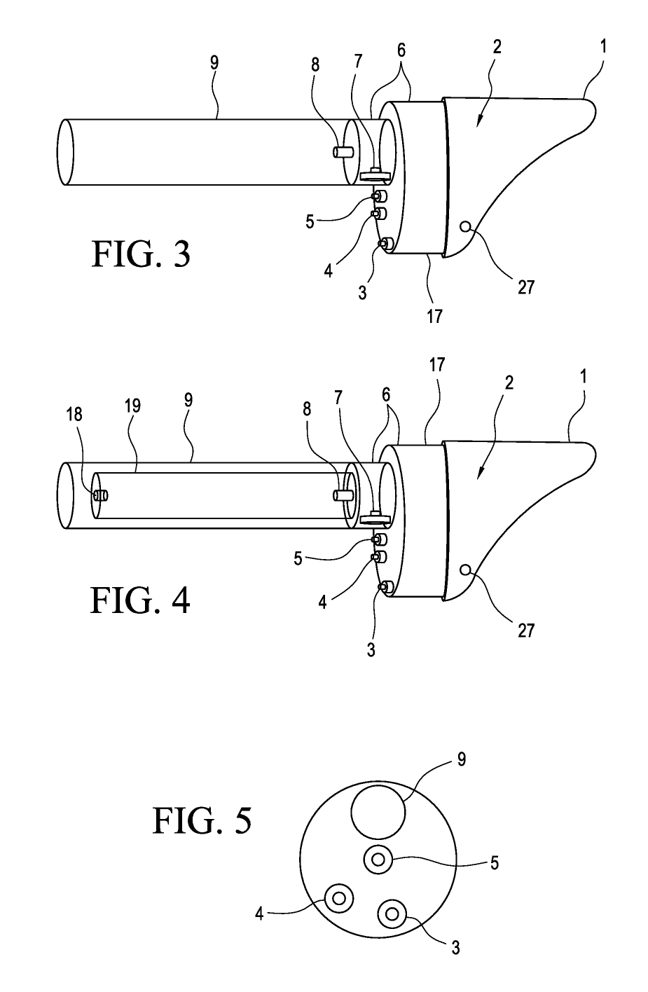

### DESCRIPTION OF THE PREFERRED EMBODIMENTS OF THE INVENTION

In essence, the present invention relates to a pulmonary delivery system 20 and/or device 20′ for inhaling micron droplets of a liquid medical product into an individual's lungs, through the lungs and into the individual's bloodstream. The system 20 and/or device 20′ include a cylinder 21 and a replaceable cartridge 29 containing a mass of about three to five milliliters of liquid medical product therein. The device also includes an atomizer 24 for producing a mist of micron droplets of a liquid medical product wherein the atomizer 24 includes a vibrating micron mesh grid 30 with a platinum or palladium surface for providing a mist with particle sizes of five microns or less and preferably about two microns. Applicant's system includes a cartridge of limited content of three to five mis to avoid a child or pet from swallowing a toxic amount of nicotine.

The vibrating micron mesh grid 30 is essentially conventional and has been used in nebulizers such as an Aerogen® Vibrator, Aerogen® Solo, and Aerogen® Ultra. Those micron mesh grids 30 are incorporated in nebulizers as an aerosol drug delivery system. For contrast, Applicant's invention utilizes a vibrating micron mesh grid 30 for producing particle sizes of about two microns with an aperture plate having about 4,800 precision holes in a grid of about ½ inch diameter. The micron mesh grid 30 vibrates at about 128,000 times per second for an average particle size of about two microns. The aperture plate should have about 4,800 precision formed holes for producing particle sizes of two microns.

In the present invention, Applicant has combined a vibratable micron mesh grid 30 with an air entrainment port 25 and a posterior entrainment port 27 by a self-inhaled breath through the mouthpiece 26 to draw air into and through an individual's lungs and into their bloodstream. The posterior entrainment port 27 further dilutes the nicotine solution and reduces coughing.

Applicant's drug delivery system 20 and device 20′ uses a breath to draw air through the air entrainment ports 25 and 27. When air drawn into the anterior entrainment port 25 and through the cylinder and around the nicotine cartridge and into the micro mesh grid and flows into the cylinder 21 and through the device 20′. The cylinder contains a pierced or ruptured cartridge from which the micron droplets are formed by the vibratable micron mesh grid for transmission into the individual's lungs and into their bloodstream. These absorbable micron droplets almost immediately satiate the craving for nicotine without effluent smoke therefore not contaminating the environment. The device 20′ may also include red and green lights that are programmed to indicate when the micron mesh grid is being vibrated.

It is presently believed that inhaling small concentrations of nicotine delivered directly into the bloodstream will relieve the symptoms of nicotine withdrawal and hopefully allow the elimination of smoking. It is presently believed that Applicant's approach relieves the desire for smoking a tobacco product and hopefully does away with the use of tobacco products in the future.

An important feature or element in a preferred embodiment of the invention relates to a microprocessor 32 that limits the inhalation of an initial and subsequent “puff” (inhaled time) to between two to four seconds. This feature causes a delay of 15 seconds prior to the next puff and should prevent a child from inhaling an excess amount of nicotine. In other words, there is a 15 second delay as a safety step to avoid an excess nicotine delivery.

Returning to a first embodiment of the invention a pulmonary delivery system for inhaling micron droplets of a liquid medical product into an individual's lungs, and into their bloodstream comprises an atomizer for producing the micron droplets of a liquid medical product wherein the atomizer includes a vibrating micron mesh grid with a platinum or palladium surface for producing a mist with particle sizes of five microns or less. As stated the micron mesh includes a mass of holes of for example 4,800 in a five millimeter diameter vibrating plate. The elongated body includes a penetrating element for rupturing or opening a cartridge and releasing the liquid medical product such as a nicotine/solution into the device. In one embodiment of the invention, a mouthpiece for delivering the medical product to a patient through an anterior air entrainment port around and through the cartridge and into the micron mesh grid.

Still further, in this embodiment, a source of electrical energy such as two double AA 1.5 volt batteries in series powers the grid whereas the inhalation of the medical product is provided solely by an individual's inhaled breath.

In the preferred embodiment of the invention, a microchip limiting the time of inhalation to between two and four seconds and is programmed that after the initial inhalation there is a 15 second delay before another breath can be taken.

A fifth embodiment of the invention involves adding a cleaning solution to each cartridge so that the medical product includes about 2% ethyl alcohol and about one percent citric acid for cleaning the grid. In addition, it should be recognized that the size, shapes and number of the holes and/or nozzles may be changed to accommodate different viscosities of solutions.

It is believed by the Applicant that if equals are substituted for equals (nicotine) and the pattern and concentrations of those addicted it should be helpful for them to quit the tobacco products that they currently use.

While the invention has been described in connection with its preferred embodiments it should be recognized that changes and modifications may be made therein without departing from the scope of the appended claims.

What is claimed is:

1. A pulmonary delivery system for inhaling micron droplets of a liquid medical product into and through an individual's lungs and into their bloodstream, said system comprising:
an elongated tubular body including an openable and closeable portion thereof for receiving a container of relatively pure medical product disposed in a saline solution, a penetrating element disposed in said elongated body for opening said container and releasing said medical product/saline solution into an airstream;
an atomizer or nebulizer for producing a mist of micron droplets from said medical product/saline solution and wherein said atomizer or nebulizer includes a vibratable micron mesh grid with a metal plate selected from the group consisting of platinum and palladium and having thousands of precision holes therein;
an entrainment port and a mouthpiece for drawing a breath of air from said entrainment port through said cylinder through said vibratable micron mesh grid and into an individual's lungs; and,
a source of electricity vibrating said micron mesh grid to produce micron droplets of said medical product/saline solution and wherein the airflow includes micron droplets is provided by the inhalation of an inhaled breath through said mouthpiece.

2. The pulmonary delivery system for inhaling micron droplets of a liquid medical product into and through an individual's lungs and into their bloodstream according to claim 1, wherein said medical product is relatively pure nicotine and an individual's intake of breath is the sole power for drawing particles into and through the individual's lungs and into their bloodstream.

3. The pulmonary delivery system for inhaling micron droplets of a liquid medical product into and through an individual's lungs and into their bloodstream according to claim 1, in which said source of electricity is two 1.5 V batteries.

4. The pulmonary delivery system for inhaling micron droplets of a liquid medical product into and through an individual's lungs and into their bloodstream according to claim 1, in which said source of electricity is two rechargeable nickel cadmium AA batteries in series.

5. The pulmonary delivery system for inhaling micron droplets of a liquid medical product into and through an individual's lungs and into their bloodstream according to claim 1, in which said vibrating micron mesh includes a platinum surface.

6. The pulmonary delivery system for inhaling micron droplets of a liquid medical product into and through an individual's lungs and into their bloodstream according to claim 5, in which said vibrating micron mesh includes a palladium surface.

7. The pulmonary delivery system for inhaling micron droplets of a liquid medical product into and through an individual's lungs and into their bloodstream according to claim 1, in which an electrical circuit connects said metal plate in said micron mesh grid to said source of electrical energy and includes two metal-oxide semiconductor field effect transistors (MOSFETs) for energizing the vibration of said metal plate.

8. The pulmonary delivery system for inhaling micron droplets of a liquid medical product into and through an individual's lungs and into their bloodstream according to claim 1, which includes a red and green light to indicate if the vibration of said micron mesh grid is off or on.

9. An inhaler for limiting an amount of an initial inspiration and to prevent a second or subsequent inspiration for a preselected time following an initial or previous inspiration, said inhaler comprising:
a cartridge containing a limited supply of a liquid pharmaceutical product or modification thereof;
means for producing a mist from said pharmaceutical product or modification thereof and wherein said means and a vibratable micron mesh grid and wherein said vibratable micron mesh includes 4,800 precision holes therein and wherein said micron mesh grid produces an average particle size of about two microns;
a source of electric energy and circuit for vibrating said micron mesh or grid and a mouthpiece for an individual to inhale through to draw particles into and through said individual's lungs and into their bloodstream.

10. The inhaler for limiting an amount of an initial inspiration and to prevent a second or subsequent inspiration for a preselected time following an initial or previous inspiration according to claim 11, in which said source of electric energy is a battery.

11. The inhaler for limiting an amount of an initial inspiration and to prevent a second or subsequent inspiration for a preselected time following an initial or previous inspiration according to claim 11, in which said source of electric energy is two 1.5 volt alkaline AA batteries in series.

12. The inhaler for limiting an amount of an initial inspiration and to prevent a second or subsequent inspiration for a preselected time following an initial or previous inspiration according to claim 1, in which a housing and a cartridge cylinder including a penetrating element for piercing or rupturing said cartridge and releasing said liquid pharmaceutical product or modification thereof into said cylinder and into said vibratable micron mesh grid.

13. The inhaler for limiting an amount of an initial inspiration and to prevent a second or subsequent inspiration for a preselected time following an initial or previous inspiration according to claim 1, in which said cartridge contains between three and five mls of a nicotine saline solution with a greatly reduced concentration of nicotine without any heating.

14. The inhaler for limiting an amount of an initial inspiration and to prevent a second or subsequent inspiration for a preselected time following an initial or previous inspiration according to claim 5, in which said cartridge contains a formulation including nicotine.

15. The inhaler for limiting an amount of an initial inspiration and to prevent a second or subsequent inspiration for a preselected time following an initial or previous inspiration according to claim 1, wherein an individual's intake of breath is the sole power for drawing particles into and through the individual's lungs and into their bloodstream.

16. The inhaler for limiting an amount of an initial inspiration and to prevent a second or subsequent inspiration for a preselected time following an initial or previous inspiration according to claim 12 which includes means for limiting an initial or subsequent inspiration to a duration of about two to four seconds and means for preventing a second and subsequent inhalation for a period of about 15 seconds.

17. The inhaler for limiting an amount of an initial inspiration and to prevent a second or subsequent inspiration for a preselected time following an initial or previous inspiration according to claim 18 which includes a microprocessor for actuating means for preventing an initial or subsequent inspiration in excess of two to four seconds and means for preventing a second or subsequent inspiration for 15 seconds activated by said microprocessor.

18. A pulmonary delivery device for inhaling micron droplets of a liquid nicotine solution into an individual's lungs and into their bloodstream, said device comprising:
an atomizer for producing a mist of micron droplets of liquid nicotine solution wherein said atomizer includes a vibratable micron mesh grid with a platinum or palladium surface and with particle sizes of about two microns;
said micron mesh grid including one or more tapered passageways extending through said micron mesh grid;
an elongated body having an openable and closeable cylinder and a cartridge disposed therein and containing up to five milliliters of nicotine solution and a penetrating element for opening said cartridge and releasing said nicotine solution into and onto said micron mesh grid;
a battery, an electrical circuit for vibrating said micron mesh grid; and
a mouthpiece for drawing a breath of air through said device and into an individual's lungs and bloodstream.

19. A medical product for reducing and/or eliminating the symptoms of withdrawal from nicotine, said product comprising a mixture of between about 97% and about 99 ¾% of a liquid saline solution and from about ¼% and about 3% of a pharmaceutical grade of nicotine plus an addition of up to about 3% of a flavor.

20. A medical product for reducing and/or eliminating the symptoms of withdrawal from nicotine, said product comprising a mixture of between about 98 ½% saline solution and about 1½% nicotine.

21. A medical product for reducing and/or eliminating the symptoms of withdrawal from nicotine according to claim 19, in which each cartridge includes a cleaning solution.

22. A medical product for reducing and/or eliminating the symptoms of withdrawal from nicotine according to claim 20, in which said cleaning solution amounts to 2% ethyl alcohol and 1% citric acid.
